(19) United States
(12) Patent Application Publication
(10) Pub. No.: US 2024/0353295 A1
(43) Pub. Date: Oct. 24, 2024

(54) QUALITY MANAGEMENT SYSTEM, OBJECT MANAGEMENT SYSTEM, AND OBJECT MANAGEMENT METHOD

(71) Applicant: ORGANO CORPORATION, Tokyo (JP)

(72) Inventors: Satoshi NAKAI, Tokyo (JP); Kyohei TSUTANO, Tokyo (JP)

(73) Assignee: ORGANO CORPORATION, Tokyo (JP)

(21) Appl. No.: 18/682,234

(22) PCT Filed: Aug. 1, 2022
(86) PCT No.: PCT/JP2022/029453

§ 371 Date: Feb. 8, 2024

(30) Foreign Application Priority Data
Aug. 10, 2021 (JP) 2021-130607

(51) Int. Cl. G01N 1/20; G01N 30/96; G01N 33/18

(52) U.S. Cl. CPC G01N 1/20; G01N 30/96; G01N 33/18

(57) ABSTRACT

An acquisition unit acquires concentration period information indicating a period of time during which impurities contained in cleaning liquid for cleaning objects are concentrated in a concentration means, a database stores as associated information object identification information uniquely assigned to the objects and cleaning period information indicating periods of time during which the objects were cleaned, and an identification unit, based on the concentration period information acquired by the acquisition unit and the associated information stored in the database, identifies the objects that were cleaned using the cleaning liquid corresponding to the cleaning liquid that was supplied to the concentration means during the concentration period indicated by the concentration period information.

### TECHNICAL FIELD

This invention relates to a quality management system, an object management system, and an object management method.

### BACKGROUND OF ART

In recent years, semiconductor devices and other electronic components are being used in various settings and have become increasingly sophisticated and highly integrated. Needless to say, high quality must be ensured in such electronic components. Generally, in order to supply the market with electronic components that meet these needs, various processes are conducted such as precision design processes, manufacturing processes including cleaning processes using ultrapure water, and rigorous inspection processes. For example, a method is considered in which the relationship between the processing conditions in each process and the quality management conditions in the subsequent process is stored in advance, the quality management conditions corresponding to the detected processing conditions are selected based on the stored relationship, and the quality of the semiconductor device is judged based on the selected quality management conditions and the detected quality (see, for example, Patent Document 1).

### PRIOR ART DOCUMENTS

### Patent Documents

Patent Document 1: Patent Publication No. 2004-296676

### SUMMARY OF THE INVENTION

### Problem to be Solved by the Invention

Quality management in the technology as described above is process-by-process quality management. This type of management makes it difficult to strictly manage quality according to the period of time during which a product was manufactured.

The purpose of the present invention is to provide a quality management system, an object management system, and an object management method that can strictly manage quality according to the period of time.

### Means for Solving the Problem

The quality management system of the present invention comprises an acquisition unit that acquires concentration period information indicating a period of time during which impurities contained in cleaning liquid for cleaning objects are concentrated by a concentration means, a database that stores object identification information uniquely assigned to the objects and cleaning period information indicating the period during which the objects were cleaned in association with each other as associated information and an identification unit that identifies objects that were cleaned using a cleaning liquid corresponding to the cleaning liquid supplied to the concentration means during the concentration period indicated by the concentration period information, based on the concentration period information acquired by the acquisition unit and the associated information stored in the database.

The object management system of the present invention also comprises a liquid quality measurement unit that measures liquid quality of cleaning liquid used to clean objects, a regulating valve that is installed in a flow path that supplies the cleaning liquid to a cleaning tank and an open/close control unit that controls opening and closing of the regulating valve based on the liquid quality measured by the liquid quality measurement unit.

The object management method of the present invention also measures liquid quality of cleaning liquid used to clean objects and controls supply of a cleaning liquid to a cleaning tank for cleaning objects using a regulating valve installed in a flow path that supplies the cleaning liquid to the cleaning tank based on liquid quality of the cleaning liquid for cleaning the objects.

### Advantageous Effects of the Invention

In the present invention, quality can be strictly managed according to the period of time.

### BRIEF DESCRIPTION OF THE DRAWINGS

FIG. 1 is a diagram showing an embodiment of a quality management system.

FIG. 2 is a table showing an example of associated information stored in a database shown in FIG. 1.

FIG. 3 is a flowchart for explaining an example of processing in the quality management system shown in FIG. 1.

FIG. 4 is a diagram showing a first embodiment of the application of the quality management system of the present invention.

FIG. 5 is a diagram showing a second embodiment of the application of the quality management system of the present invention.

FIG. 6 is a diagram showing an embodiment of an object management system of the present invention.

FIG. 7 is a diagram showing an example of decision criteria and control details performed by the open/close control unit shown in FIG. 6.

FIG. 8 is a flowchart for explaining an example of an object management method in the object management system shown in FIG. 6.

FIG. 9 is a diagram showing a first embodiment of the application of the object management system.

FIG. 10 is a diagram showing a second embodiment of the application of the object management system.

### DESCRIPTION OF THE EMBODIMENTS

Embodiments of the present invention are next described with reference to the drawings.

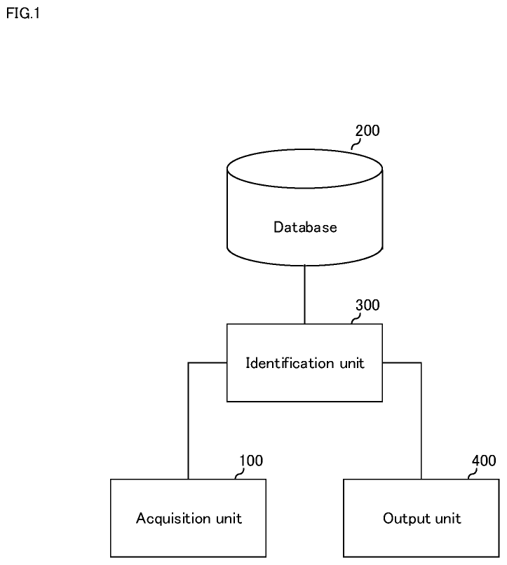

FIG. 1 shows an embodiment of a quality management system. The quality management system in this embodiment has acquisition unit 100, database 200, identification unit 300, and output unit 400, as shown in FIG. 1.

Acquisition unit 100 acquires concentration period information indicating the period of time during which cleaning liquid for cleaning objects is concentrated by a concentration means that concentrates impurities contained in the cleaning liquid. Here, the objects are, for example, electronic components such as semiconductor devices. The concentration means is an adsorbent that adsorbs metallic impurities such as metal ions as impurities. The concentration means includes, for example, monolithic organic porous media. Concentration period information is recorded and maintained for each adsorbent. The concentration period information consists of, for example, adsorbent identification information assigned in advance to each adsorbent through which the cleaning liquid is passed, and concentration period information indicating the period (date and time) during which the cleaning liquid was passed through that adsorbent, and is stored in a database (not shown, but which may be database 200 shown in FIG. 1) as a flow-through record. Acquisition unit 100 may acquire the concentration period information from a database in which flow-through records are stored. Acquisition unit 100 may also acquire the data based on external operations (inputs) by the operator. The cleaning liquid supplied to the concentration means to concentrate impurities is at least a portion of the cleaning liquid that cleans the objects, and it is not necessary to supply all of the cleaning liquid that cleans the objects. The concentration means can also be a means that uses a method of concentrating the substances to be analyzed by installing multiple stages of reverse osmosis membrane devices or electro-deionization devices as described in JP-2004-77299. The concentration means can also be a means that uses a method of using a permeable membrane as described in International Publication No. 2012-073566. The concentration means can also be a means that uses a method of concentrating on the surface of the sample as described in JP-2004-101408.

Database 200 stores as associated information the object identification information assigned uniquely to objects and the cleaning period information indicating the period during which objects were cleaned.

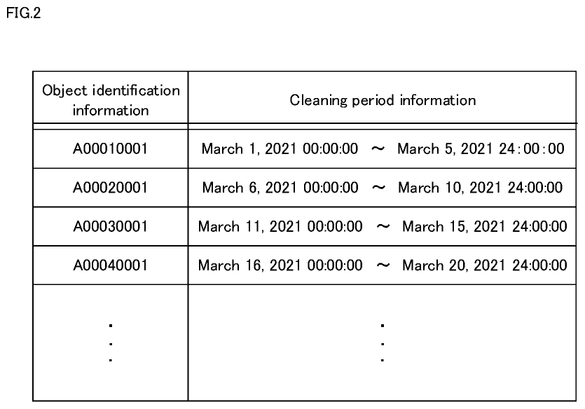

FIG. 2 shows an example of associated information stored in database 200 shown in FIG. 1. The associated information stored in database 200 shown in FIG. 1 is information that associates object identification information with cleaning period information, as shown in FIG. 2. The object identification information is preassigned identification information assigned uniquely in advance to objects to be cleaned with the cleaning liquid. This object identification information can be used to identify the objects. The object identification information should be sufficient to identify the objects from each other. The object identification information may be, for example, a combination of several alphabet characters and numbers. The cleaning period information is information indicating the date and time of the start of cleaning and the date and time of the end of cleaning so that the period (e.g., time period) during which an object was cleaned can be identified. For example, as shown in FIG. 2, the object identification information “A00010001” is associated with the cleaning period information “March 1, 2021 00:00:00 to Mar. 5, 2021 24:00:00.” This indicates that the object to which the object identification information “A00010001” is assigned was cleaned with a cleaning liquid from 00:00:00 on Mar. 1, 2021 to 24:00:00 on Mar. 5, 2021. In addition, the object identification information “A00020001” is associated with the cleaning period information “Mar. 6, 2021 00:00:00 to Mar. 10, 2021 24:00:00.” This indicates that the object to which the object identification information “A00020001” is assigned was cleaned with a cleaning liquid from 00:00:00 on Mar. 6, 2021 to 24:00:00 on Mar. 10, 2021. In addition, the object identification information “A00030001” is associated with the cleaning period information “Mar. 11, 2021 00:00:00 to Mar. 15, 2021 24:00:00.” This indicates that the object to which the object identification information “A00030001” is assigned was cleaned with a cleaning liquid from 00:00:00 on Mar. 11, 2021 to 24:00:00 on Mar. 15, 2021. In addition, the object identification information “A00040001” is associated with the cleaning period information “Mar. 16, 2021 00:00:00 to Mar. 20, 2021 24:00:00.” This indicates that the object to which the object identification information “A00040001” is assigned was cleaned with a cleaning liquid from 00:00:00 on Mar. 16, 2021 to 24:00:00 on Mar. 20, 2021. These associations are stored in database 200 based on operations (inputs) made by the operator after the cleaning of each object is completed. After each object has been cleaned, information indicating when the cleaning started and when the cleaning ended may be entered and stored in database 200. Information indicating the start time of cleaning may be entered and stored in database 200 when the cleaning of each object is started, and information indicating the end time of cleaning may be entered and stored in database 200 when the cleaning of each object is finished.

Identification unit 300 identifies an object based on the concentration period information acquired by acquisition unit 100 and the associated information stored in database 200. Specifically, identification unit 300 searches database 200 for the object identification information associated with the cleaning period information that indicates the cleaning period corresponding to the concentration period information acquired by acquisition unit 100. Identification unit 300 then identifies the object to which the retrieved object identification information is assigned. The relationship between the concentration period and the cleaning period depends on the distance (pipe distance) between the location where acquisition unit 100 is located and the location where the object is to be cleaned in the system and the flow rate in the pipes between them (pipe flow rate). For example, if the pipe distance is 100 m and the pipe flow rate is 2 m/sec, the cleaning period is 50 seconds after the concentration period. In other words, identification unit 300 calculates the cleaning period based on the period indicated by the concentration period information acquired by acquisition unit 100 and the processing time obtained from the specifications of the system. Then, identification unit 300 identifies the object by searching database 200 for the object identification information associated with the cleaning period using the cleaning period information indicating the calculated cleaning period as a search key. Identification unit 300 thereby identifies the object that was cleaned using the cleaning liquid corresponding to the cleaning liquid that was at least partially supplied to the concentration means during the concentration period indicated by the concentration period information acquired by acquisition unit 100.

Output unit 400 outputs the object identification information indicating the object identified by identification unit 300. Output unit 400 may display the object identification information. Output unit 400 may also transmit the object identification information to other devices. The output mode of output unit 400 is not limited.

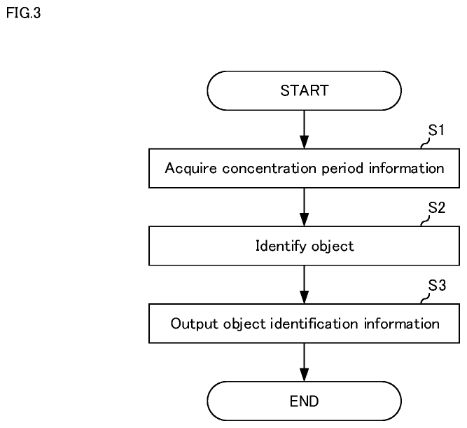

The following is a description of the process in the quality management system shown in FIG. 1. FIG. 3 is a flowchart for explaining an example of processing in the quality management system shown in FIG. 1. First, acquisition unit 100 acquires concentration period information (Step S1). The timing at which acquisition unit 100 acquires the concentration period information may be a predetermined timing (e.g., periodic). The timing at which acquisition unit 100 acquires the concentration period information may be based on an external request based on the results of a series of retrospective water quality analyses. Acquisition unit 100 may acquire the concentration period information from a database in which concentration period information is stored. Acquisition unit 100 may also acquire the concentration period information based on external operations (inputs). Acquisition unit 100 may also request the concentration period information from other devices that can communicate with acquisition unit 100 and then receive and acquire the concentration period information sent from the other devices.

Identification unit 300 then calculates the cleaning period according to the concentration period indicated by the concentration period information acquired by acquisition unit 100. Identification unit 300 identifies the object by searching database 200 for the object identification information associated with the cleaning period information indicating the calculated cleaning period (Step S2). Output unit 400 then outputs object identification information indicating the object identified by identification unit 300 (Step S3). Output unit 400 may output information that can identify the object identified by identification unit 300. Here, output unit 400 is not limited to outputting object identification information.

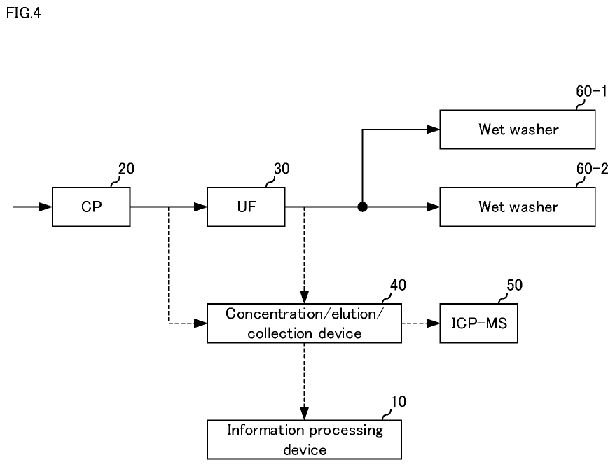

The following is an example of the application of the quality management system of the present invention. FIG. 4 shows a first embodiment of the application of the quality management system of the present invention. The system shown in FIG. 4 is a system in which ultrapure water that is cleaning water is supplied to wet washers 60-1 and 60-2 (points of use), which are semiconductor cleaning equipment, via CP 20 that is a non-regenerative ion exchange device and UF 30 that is an ultrafiltration membrane device, in an ultrapure water production facility. Wet washers 60-1 and 60-2 can be cleaning tanks. Ultrapure water supplied to CP 20 is first supplied from a pre-treatment system of an ultrapure water production facility to a primary pure water production system, is treated in the primary pure water production system, and then supplied. The broken lines in FIG. 4 show the routes of control signals or the flow path of water that is sampled from a portion of the ultrapure water, which is the cleaning water to be supplied to the point of use, to test the quality of the ultrapure water. A valve for branching may be provided at the point where the cleaning water is branched off to concentration/elution/collection device 40, and the opening and closing of the valve may be controlled.

Concentration/elution/collection device 40 is equipped with the above-described adsorbent which is the concentration means and thus adsorbs and obtains impurities in the outlet water of CP 20 or UF 30. Concentration/elution/collection device 40 elutes the adsorbed impurities by passing eluent through the adsorbent that has adsorbed impurities and collects the eluent that contains the eluted impurities. Eluents used here include, for example, nitric acid, acidic aqueous solutions such as hydrochloric acid and sulfuric acid, or alkaline aqueous solutions of organic alkalis such as trimethylhydroxyammonium and tetramethylammonium hydroxide (TMAH). A collection bottle may be used as a collection container for collecting the eluted impurities. ICP-MS 50 is a device that measures the amount of impurities in the collected eluent and calculates the impurity concentration. Information processing device 10 has acquisition unit 100, database 200, identification unit 300, and output unit 400.

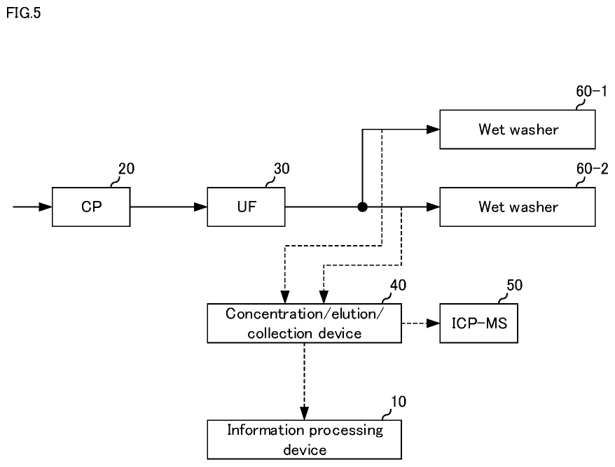

FIG. 5 shows a second embodiment of the application of the quality management system of the present invention. The embodiment shown in FIG. 5 differs from the embodiment shown in FIG. 4 in that the water that is passed through the adsorbent provided in concentration/elution/collection device 40 is water that immediately precedes being supplied to wet washer 60-1 or to wet washer 60-2. The position at which the cleaning liquid is passed through the adsorbent can be any position after the outlet of CP 20. A valve for branching may be provided at the point where the cleaning water is branched off to the concentration/elution/collection unit 40, and the opening and closing of the valve may be controlled. In FIG. 4, the case of two wet washers 60-1 and 60-2 is used as an example, but the number is not limited to that number.

Thus, in the quality management system of the present invention, acquisition unit 100 acquires concentration period information indicating the period of time during which the cleaning liquid for cleaning an object was passed through the adsorbent. Identification unit 300 calculates the cleaning period based on the period indicated by the concentration period information. Identification unit 300 identifies the object by searching database 200 for the object identification information using as a search key the cleaning period information that indicates the calculated cleaning period. This procedure allows for strict management of quality according to the period of time the object was cleaned.

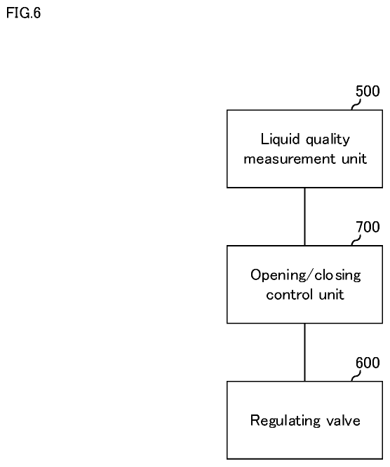

FIG. 6 shows an embodiment of an object management system of the present invention. The object management system in this embodiment has liquid quality measurement unit 500, regulating valve 600, and opening/closing control unit 700, as shown in FIG. 6.

Liquid quality measurement unit 500 measures the liquid quality of the cleaning liquid used to clean an object. Liquid quality measurement unit 500 measures the amount (e.g., concentration) of impurities in the cleaning liquid. When impurities in the cleaning liquid are concentrated using an adsorbent, liquid quality measurement unit 500 measures the concentration of impurities adsorbed in the adsorbent. When the adsorbent adsorbs metallic impurities (e.g., metal ions) as impurities, liquid quality measurement unit 500 may elute the metal ions adsorbed in the adsorbent using an eluent and then measure the concentration of the metal ions in the eluent that has passed through the adsorbent. The adsorbent can be a monolithic organic porous material. Liquid quality measurement unit 500 can also be ICP-MS 50 shown in FIGS. 4 and 5. In other words, liquid quality measurement unit 500 is a device that, for example, uses an eluent to elute impurities adsorbed in an adsorbent, collects the eluent, measures the amount of impurities in the collected eluent, and calculates the amount of impurities in the cleaning liquid. Here, based on the results measured by liquid quality measurement unit 500, identification unit 300 shown in FIG. 1 begins to identify the object. For example, if the amount of impurities measured by liquid quality measurement unit 500 exceeds a predetermined amount, it becomes necessary to identify the object that was cleaned using that cleaning liquid, and identification unit 300 therefore begins the identification of the object.

Regulating valve 600 is a regulating valve installed in the flow path that supplies cleaning liquid to the cleaning tank. Regulating valve 600 opens and closes to control the supply of cleaning liquid downstream of regulating valve 600 in that flow path. Regulating valve 600 can, for example, control the supply of cleaning liquid to the point where an object is to be cleaned with the cleaning liquid. Regulating valve 600 opens and closes based on control signals from opening/closing control unit 700. The specific location of regulating valve 600 is described below.

Opening/closing control unit 700 controls the opening and closing of regulating valve 600 based on the liquid quality (i.e., the amount of impurities in the cleaning liquid) measured by liquid quality measurement unit 500. Opening/closing control unit 700 opens regulating valve 600 when the liquid quality measured by liquid quality measurement unit 500 meets a predetermined reference value (i.e., when the liquid quality is below the predetermined reference value). If the liquid quality measured by liquid quality measurement unit 500 does not meet the reference value (i.e., exceeds the predetermined reference value), open/close control unit 700 closes regulating valve 600. If there is no significant fluctuation in the water flow rate per unit time to the adsorbent, opening/closing control unit 700 may control the opening and closing of regulating valve 600 based on the amount of impurities in the eluent measured by liquid quality measurement unit 500. In addition, opening/closing control unit 700 may control the opening and closing of regulating valve 600 using values that can determine the level of water quality of the cleaning liquid from the values measured by liquid quality measurement unit 500.

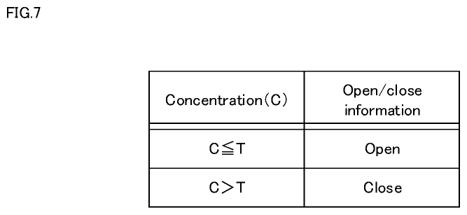

FIG. 7 shows an example of decision criteria and control details performed by opening/closing control unit 700 shown in FIG. 6. In the example shown in FIG. 7, the reference value for decision is the value of impurity concentration. As shown in FIG. 7, a case where the concentration of impurities is below a preset threshold value T is associated with the open/close information “open.” Based on this association, opening/closing control unit 700 opens regulating valve 600 if the liquid quality (concentration of impurities) measured by liquid quality measurement unit 500 is equal to or less than the threshold value T, as this case meets the reference value. In this way, cleaning liquid that meets the reference value can be supplied to the point of use (cleaning tank). If the liquid quality (concentration of impurities) measured by the liquid quality measurement unit 500 exceeds the threshold value T, which is the reference value, opening/closing control unit 700 assumes that this case does not satisfy the reference value and closes regulating valve 600. In this way, it is possible to avoid supplying the point of use (cleaning tank) with cleaning liquid that does not meet the reference value.

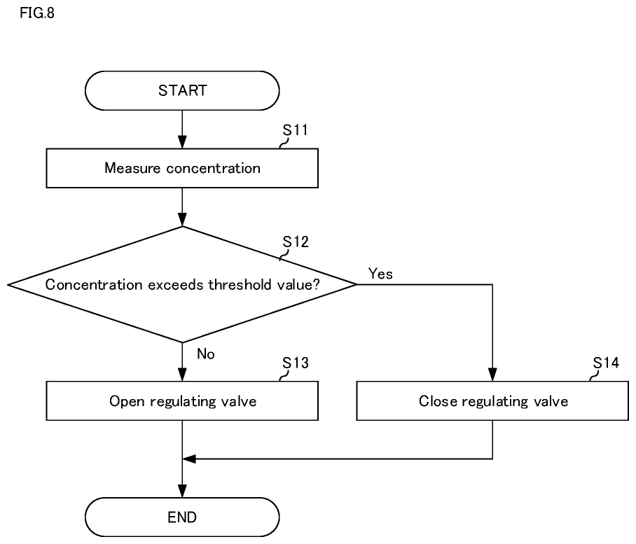

An object management method in the object management system shown in FIG. 6 is next described. FIG. 8 is a flowchart for explaining an example of an object management method in the object management system shown in FIG. 6. Here, liquid quality measurement unit 500 will be described using the case of measuring the concentration of impurities in the cleaning liquid as an example.

First, liquid quality measurement unit 500 measures the concentration of impurities in the cleaning liquid for cleaning an object (Step S11). Opening/closing control unit 700 then determines whether the concentration of impurities measured by liquid quality measurement unit 500 exceeds a preset threshold value (Step S12). If it is determined that the concentration of impurities measured by liquid quality measurement unit 500 does not exceed the threshold value, opening/closing control unit 700 opens regulating valve 600 (Step S13). If regulating valve 600 is already open at this time, the opening/closing control unit 700 keeps regulating valve 600 in the open state. On the other hand, if it is determined that the concentration of impurities measured by liquid quality measurement unit 500 exceeds the threshold value in Step S12, opening/closing control unit 700 closes regulating valve 600 (Step S14). If regulating valve 600 is already closed at this time, opening/closing control unit 700 keeps regulating valve 600 in the closed state.

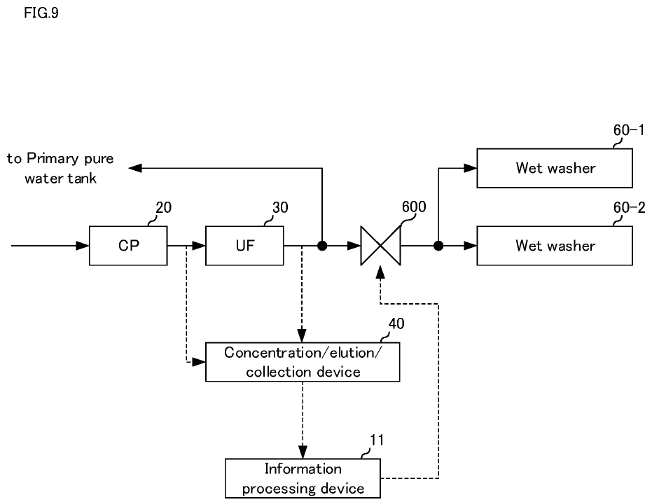

The following is an example of the application of the object management system. FIG. 9 shows a first embodiment of the application of the object management system. The embodiment shown in FIG. 9 is a system in which ultrapure water that is cleaning water is supplied by way of CP 20 that is a non-regenerative ion exchange device, UF 30 that is an ultrafiltration membrane device, and regulating valves 600 in the ultrapure water production facility located downstream of the primary pure water tank where primary pure water is stored to wet washers 60-1 and 60-2 (points of use). CP 20, UF 30, and wet washers 60-1 and 60-2 are each the same as those shown in FIG. 4. Components such as a pump, heat exchanger, and UV oxidizer may be provided between the primary pure water tank and CP 20. Regulating valve 600 is described using FIG. 6 and controls the supply of water from UF 30 to wet washers 60-1 and 60-2. The ultrapure water supplied to CP 20 is supplied from a liquid production and supply facility located upstream. Liquid production and supply facilities are also facilities that produce ultrapure water. The broken lines in FIG. 9 show the paths of water flow or control signals for testing the quality of ultrapure water, which is the cleaning water. A valve for branching may be provided at the point where the cleaning water is branched off to the concentration/elution/collection device 40, and the opening and closing of the valve may be controlled. Return piping is provided to return water from UF 30 to the primary pure water tank.

Concentration/elution/collection device 40 is the same as that shown in FIG. 4 and acquires impurities in the outlet water of CP 20 or UF 30 by adsorption. Concentration/elution/collection device 40 elutes adsorbed impurities by passing eluent through the adsorbent that has adsorbed impurities and collects eluent containing the eluted impurities. Information processing device 11 has liquid quality measurement unit 500 and opening/closing control unit 700. Liquid quality measurement unit 500 measures the liquid quality of the cleaning liquid based on the eluent collected by concentration/elution/collection device 40. Information processing unit 11 and regulating valve 600 constitute the object management system of the present invention.

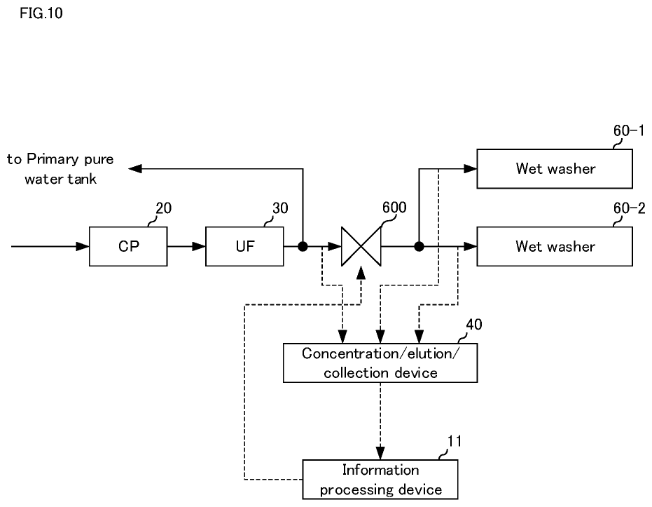

FIG. 10 shows a second embodiment of the application of the object management system. The embodiment shown in FIG. 10 differs from the embodiment shown in FIG. 9 in that the water that is passed through the adsorbent provided in concentration/elution/collection device 40 is, in addition to the water that flows from UF 30 to regulating valve 600, water that immediately precedes being supplied to wet washer 60-1 or water that immediately precedes being supplied to wet washer 60-2. The position at which the cleaning liquid is passed through the adsorbent can be any position that follows the outlet of CP 20. Valves for branching may be provided at points where the cleaning liquid is branched off to concentration/elution/collection unit 40, and the opening and closing of these valves may be controlled. In FIG. 10, a case of two wet washers 60-1 and 60-2 is used as an example, but the number is not limited to that number.

Thus, in the object management system, liquid quality measurement unit 500 measures the liquid quality of the cleaning liquid used to clean objects. Based on the liquid quality measured by liquid quality measurement unit 500, opening/closing control unit 700 controls the opening/closing of regulating valve 600 provided in the flow path that supplies cleaning liquid to the cleaning points where semiconductor devices are cleaned. This allows for real-time quality management based on the condition of the cleaning solution.

The liquid (water) to be measured is not limited to ultrapure water, but can also be a chemical solution such as IPA (isopropyl alcohol), PGMA (polyglycerol methacrylate), and PGMEA (propylene glycol monomethyl ether acetate).

Although described above by allocating each function (processing) to a respective component, these assignments are not limited to those described above. In addition, as for the configuration of the components, the above-described embodiments are merely examples, and the present invention is not limited thereto. Further, the present invention may be a combination of the embodiments.

While the present invention has been described with reference to the embodiments, the present invention is not limited to the above embodiments. Various changes within the scope of the present invention that will be understood by those skilled in the art can be made in the configuration and details of the present invention.

This application claims priority based on JP 2021-130607, filed Aug. 10, 2021, and incorporates all of its disclosure herein.

1. A quality management system, comprising:
an acquisition unit that acquires concentration period information indicating a period of time during which impurities contained in cleaning liquid for cleaning objects are concentrated by a concentration means;
a database that stores object identification information uniquely assigned to the objects and cleaning period information indicating the period during which the objects were cleaned in association with each other as associated information; and
an identification unit that identifies objects that were cleaned using a cleaning liquid corresponding to the cleaning liquid supplied to the concentration means during the concentration period indicated by the concentration period information, based on the concentration period information acquired by the acquisition unit and the associated information stored in the database.

2. The quality management system according to claim 1, further comprising:
a liquid quality measuring unit that measures an amount of impurities concentrated by the concentration means, wherein
the identification unit begins identifying the object based on the results measured by the liquid quality measurement unit.

3. The quality management system according to claim 1, wherein
the concentration means is supplied with the cleaning liquid that has passed through a non-regenerative ion exchange device provided on a flow path of the cleaning liquid.

4. The quality management system of claim 1, wherein
the concentration means concentrates metallic impurities as the impurities.

5. The quality management system according to claim 4, wherein
the concentration means is a monolithic organic porous material.

6. An object management system, comprising:
a liquid quality measurement unit that measures liquid quality of cleaning liquid used to clean objects;
a regulating valve that is installed in a flow path that supplies the cleaning liquid to a cleaning tank; and
an open/close control unit that controls opening and closing of the regulating valve based on the liquid quality measured by the liquid quality measurement unit.

7. The object management system according to claim 6, wherein
the liquid quality measurement unit measures the liquid quality of the cleaning liquid that has passed through a non-regenerative ion exchange device provided on a flow path of the cleaning liquid.

8. The object management system according to claim 6, wherein
the opening/closing control unit opens the regulating valve when the liquid quality measured by the liquid quality measurement unit meets a reference value and closes the regulating valve when the liquid quality measured by the liquid quality measurement unit does not meet the reference value.

9. An object management method that controls supply of a cleaning liquid to a cleaning tank for cleaning objects using a regulating valve installed in a flow path that supplies the cleaning liquid to the cleaning tank based on liquid quality of the cleaning liquid for cleaning the objects.
